(19) United States
(12) Patent Application Publication
(10) Pub. No.: US 2022/0265695 A1
(43) Pub. Date: Aug. 25, 2022

(54) OPTHALMIC COMPOSITIONS COMPRISING VISCOSIFYING POLYMERS AND NUCLEIC ACIDS

(71) Applicant: ProQR Therapeutics II B.V., Leiden (NL)

(72) Inventors: Gerardus Johannes PLATENBURG, Leiden (NL); Elisabeth Laurentina Wilhelmina Maria VAN MIERLO, Leiden (NL); Aliye Seda YILMAZ-ELIS, Leiden (NL)

(21) Appl. No.: 17/629,731

(22) PCT Filed: Jul. 24, 2020
(86) PCT No.: PCT/EP2020/070897

§ 371 Date: Jan. 24, 2022

(30) Foreign Application Priority Data
Jul. 26, 2019 (EP) 19188681.1
Sep. 20, 2019 (EP) 19198642.1

(51) Int. Cl. A61K 31/7088; A61K 47/38; A61K 9/00

(52) U.S. Cl. CPC A61K 31/7088; A61K 47/38; A61K 9/0048

(57) ABSTRACT

The invention relates to ophthalmic compositions comprising: i) a nucleic acid molecule, preferably an antisense oligonucleotide, such as an single-stranded antisense oligonucleotide that modulates splice modulation or prevention of RNA toxicity due to trinucleotide repeats in a target RNA molecule, or a gapmer that induces breakdown of a target RNA molecule after formation of a double-stranded RNA/gapmer complex; and ii) a viscosifying polymer. The ophthalmic compositions are for topical administration in the eye of a mammalian subject suffering from a corneal disease, such as a hereditary corneal dystrophy. The viscosifying polymer in the compositions of the invention allows the entry of the nucleic acid molecule to the different layers of the cornea: the corneal epithelium, Bowman's membrane, stroma, Dua's layer, the Descemet's membrane and/or the corneal endothelium.

### FIELD OF THE INVENTION

The present invention relates to the field of medicine, in particular to the field of preventing and treating genetic eye disorders. More in particular, the present invention relates to methods and means for the prevention and/or treatment of genetic diseases afflicting the cornea.

### BACKGROUND OF THE INVENTION

The cornea is a transparent front part of the eye that covers the iris, pupil and anterior chamber. The cornea, with the anterior chamber and lens, refracts light, with the cornea accounting for approximately two-thirds of the eye's total optical power. While the cornea contributes most of the eye's focusing power, its focus is fixed. Accommodation, or refocusing of light to better view nearby objects, is accomplished by changing the geometry of the lens. Because transparency is of prime importance, the healthy cornea does not have or need blood vessels within it. Instead, oxygen dissolves in tears and then diffuses throughout the cornea to keep it healthy. Similarly, nutrients are transported via diffusion from the tear fluid through the outside surface and the aqueous humor through the inside surface. Transparency, avascularity, the presence of immature resident immune cells, and immunologic privilege makes the cornea a very special tissue. The human cornea (see FIG. 1) has five layers, from the anterior to the posterior: corneal epithelium, Bowman's layer, corneal stroma, Descemet's membrane and corneal endothelium. A sixth layer is sometimes referred to as Dua's layer and is located between the stroma and the Descemet's membrane. At the anterior side, the corneal epithelium is covered by a thin fluid layer, the so-called pre-corneal tear film, consisting of a superficial lipid layer, a central aqueous layer and an inner mucus layer. The corneal endothelium is a squamous or low cuboidal monolayer of mitochondria-rich cells separating the corneal stroma from the anterior chamber fluid. These cells are responsible for regulating fluid and solute transport between the aqueous and corneal stromal compartments, and function as a pump, providing the cornea with the correct hydration to ensure transparency of this tissue. The clarity of the cornea depends on an ordered lamellar collagen structure and relative hydration which requires endothelial cell density of at least 1000 cells/mm². The corneal endothelium is bathed by aqueous humor, not by blood or lymph. Unlike the corneal epithelium, the cells of the endothelium do not regenerate. Instead, they stretch to compensate for dead cells that reduces the overall density of the endothelium and which affects fluid regulation. If the endothelium can no longer maintain a proper fluid balance, stromal swelling due to excess fluids and subsequent loss of transparency will occur and this may cause corneal edema and interference with the transparency of the cornea and thus impairing the image formed. The corneal endothelium is responsible for maintenance of corneal clarity by a continual process that prevents excessive hydration of cornea from an influx of cations and water molecules into the collagenous corneal stroma, generally referred to as ‘deturgescence’. The most common corneal disorders are corneal abrasion, corneal dystrophy, corneal ulcer, corneal neovascularization, keratitis, and keratoconus. There are about twenty genetic corneal dystrophies that are grouped based on the layers they affect. These are:

1) Epithelial and Sub-Epithelial Dystrophies:
Epithelial basement membrane dystrophy
Epithelial recurrent erosion dystrophies
Subepithelial mucinous corneal dystrophy
Lisch corneal dystrophy
Meesmann corneal dystrophy
Gelatinous drop-like corneal dystrophy

2) Bowman Layer Dystrophies:
Reis-Bucklers corneal dystrophy
Thiel-Behnke corneal dystrophy
TGFB1 corneal dystrophies
Lattice corneal dystrophy
Granular corneal dystrophy (type 1 and type 2)

3) Stromal Corneal Dystrophies that affect the stroma and may progress into other layers:
Macular corneal dystrophy
Schnyder chrystalline corneal dystrophy
Congenital stromal corneal dystrophy
Fleck corneal dystrophy
Posterior amorphous corneal dystrophy
Central cloudy dystrophy of Francois
Pre-Descemet corneal dystrophy

4) Posterior Corneal Dystrophies that affect the Descemet's membrane and the endothelium:
Congenital hereditary endothelial dystrophy
Fuchs' Endothelial Corneal Dystrophy (FECD)
Posterior polymorphous endothelial dystrophy
X-linked endothelial corneal dystrophy

The most common dystrophy is FECD, which is an inherited, corneal endothelial degeneration disorder associated with the presence of corneal guttae that are microscopic collagenous accumulations under the corneal endothelial layer. After the age of 40, up to 5% of US adults exhibit corneal guttae. The presence of guttae is indicative of FECD but generally represents mild disease that is completely asymptomatic. Advanced (severe) disease develops in a small proportion of patients with guttae. Advanced FECD is characterized by extensive guttae, endothelial cell loss, corneal edema, corneal clouding and consequential vision loss due to corneal edema and clouding. Corneal edema, clouding and subsequent vision loss are a direct consequence of endothelial cell degeneration and loss of deturgescence. Vision loss due to FECD is the most frequent indication requiring full thickness corneal transplantation (penetrating keratoplasty), accounting for greater than 14,000 procedures annually in the US alone. No other treatments are available for FECD.

Although corneal transplantation is a largely successful treatment it has the disadvantage that it is invasive and associated with an approximate 30% rejection rate, which is not dissimilar to other solid organ allografts. An alternative approach in which just the corneal endothelium is replaced (endokeratoplasty) can also be carried out, but only by very experienced surgeons. Both interventions suffer from lack of donor material, either transplantable corneal buttons or corneal derived endothelial cells derived from donor corneas. FECD is also a risk for other procedures such as cataract surgery and is contraindicated for refractive surgery such as Laser-Assisted in situ Keratomileusis (LAISK) as these techniques lead to additional corneal endothelial cell loss.

There remains an unmet medical need to treat patients suffering from, or that are at risk of developing corneal diseases by providing proper alternatives for transplantation. One of such treatments is the use of (single-stranded antisense) RNA and/or RNA/DNA oligonucleotides that target the (pre-) mRNA of the mutated gene that causes the genetic disorder, thereby influencing the splicing machinery (e.g. by exon skipping, for instance by skipping the mutated exon, or skipping an aberrantly spliced exon), by preventing or reducing RNA toxicity due to trinucleotide repeats (TNR's) present in the RNA, or by targeting the RNA for downregulation of transcript expression through nuclease-dependent breakdown of the double-stranded target/oligonucleotide complex. WO2017/060317 discloses the use of single-stranded antisense oligonucleotides that target intronic CAG repeats to prevent the formation of RNA foci, especially for the treatment of genetic disorders such as FECD. US2009/163432A1 discloses the use of a nucleic acid molecule (siRNA) to downregulate the expression of connexin 43 transcripts to prevent the reduction of corneal endothelial cells. WO2004/108945 discloses the use of a double-stranded RNA oligonucleotide targeting the ICAM-1 transcript for the preservation of corneal explants.

Notably, the cornea is a strong barrier against influences from outside the eye, and it remains a challenge to penetrate the outer layers of the cornea to reach the different layers, such as the epithelium, Bowman's membrane, the stroma and the endothelium, especially when these are afflicted by genetic defects (such as FECD, exemplified above) that should be reversed to ensure proper sight. Because of its structure the cornea provides a strong barrier against influences from outside. The continuous replacement of the tear film makes that drugs are easily washed out and it remains a challenge to deliver drugs that pass the tear film as well as the anterior layers of the cornea. In any case, the epithelium is the main limitation for intracorneal drug delivery. One of the ways to improve drug delivery to the eye is the use of penetration enhancers. These are considered compounds capable of enhancing drug permeability across ocular membranes, such as those in the cornea, and acting predominantly on the corneal epithelium. Drug penetration enhancement can be achieved by inclusion of agents capable of modifying the tear film, mucous layer and ocular membranes. A further strategy for enhancing drug penetration into the eye can be achieved by energy-driven means, where a small electrical current is used (iontophoresis) or ultrasound is employed to drive the drug to enter the ocular tissues. Moiseev and co-workers reviewed the state of the art in the use of penetration enhancers in drug delivery to the eye (Moiseev et al. Penetration enhancers in ocular drug delivery. Pharmaceutics 2019, 11, 321), describing the wide variety of lubricants (carboxymethylcellulose, hydroxypropyl methylcellulose (or hypromellose (HPMC)), carbomer gels), xanthan gum, phospholipids, artificial tears, etc. and their use in drug delivery through topical application, as well the development of theoretical models that predict the permeability of the cornea for different solutes. Examples of penetration enhancers that have been used in the art are cyclodextrins, chelating agents (e.g. EDTA, EGTA, BAPTA and EDDS), crown ethers, surfactants (e.g. plant surfactants (digitonin), benzalkonium chloride, Tween-20, Tween-80, Span-60, Brij-35, -78 and -98), bile acids and bile salts (e.g. deoxycholate, glycocholate, taurodeoxycholate, cell-penetrating peptides, and other amphiphilic compounds. EP2526923B1 discloses an ophthalmic gel comprising gatifloxacin, carbomer, sodium hyaluronate and hypromellose for the treatment of eye infections. WO2012/136969 discloses aqueous compositions suitable for topical administration to the eye that contain at least one polymeric ophthalmic lubricant, such as hyaluronate, carbomer gel or hypromellose, together with a water-soluble analgesic, predominantly for the treatment of dry eye or blepharitis. The use of a composition comprising an oligonucleotide and poloxamer 407 gel was investigated and it was found that ocular adsorption was only achieved via the sclera and conjunctiva but not through the cornea (Bochot et al. Comparison of the ocular distribution of a model oligonucleotide after topical instillation in rabbits of conventional and new dosage forms. J Drug Targeting. 1998. 6(4):309-313). WO2011/140194 discloses ophthalmic compositions comprising hyperbranched polyesters that increase corneal permeation of the active agent in the composition. A variety of drug delivery vehicles for penetration of the cornea were described (Johnson L N. et al. Cell-penetrating peptide for enhanced delivery of nucleic acids and drugs to ocular tissues including retina and cornea. Mol Ther 2018. 16(1):107-114; WO2014/039012; Ludwig A. The use of mucoadhesive polymers in ocular drug delivery. 2005. Adv Drug Del Rev 57:1595-1639).

It is noted that the prior art is silent on how to deliver nucleic acid molecules such as single-stranded gapmers and other types of antisense oligonucleotides (AONs) through the protecting layers to reach the corneal endothelium to treat and/or prevent the genetic disorders affecting the deeper layers of the cornea. It is an object of the present invention to provide methods (through topical administration) and means (ophthalmic compositions comprising a nucleic acid molecule as the active ingredient+a penetration enhancer or viscosifying lubricant/polymer to support the transfer of the active ingredient through the different layers of the cornea) to prevent and/or treat genetic eye diseases (preferably in human subjects) that affect the variety of layers within the cornea.

### SUMMARY OF THE INVENTION

The present invention relates to an ophthalmic composition for the treatment and/or prevention of a disorder of the cornea, said composition comprising: i) a nucleic acid molecule, ii) a viscosifying polymer, and iii) a solvent, wherein the nucleic acid molecule is at least partially complementary to, and capable of binding a target (pre-) mRNA molecule, and wherein the viscosifying polymer enables the nucleic acid molecule to penetrate through the layers within the cornea after topical administration of the composition. Preferably, the viscosifying polymer is selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine. Most preferred is HPMC. The nucleic acid and the viscosifying polymer are preferably mixed with the solvent to form a homogeneous mixture that can be topically applied to the cornea, preferably as drops or as a film. The disorder is preferably a genetic disorder, such as a hereditary corneal dystrophy, wherein the target (pre-) mRNA molecule is the cause of the genetic disorder. In one embodiment the nucleic acid molecule is a single-stranded antisense oligonucleotide (AON) that modulates splicing of the target pre-mRNA, or that prevents or reduces RNA toxicity. In another embodiment the nucleic acid molecule is a gapmer that downregulates expression of the target (pre-) mRNA. Preferably, the disorder is a dystrophy affecting the corneal epithelium, the Bowman's layer, the corneal stroma, the Descemet's membrane and/or the corneal endothelium, preferably in a human subject.

In one particular aspect, the invention relates to an ophthalmic composition according to the invention for use in the treatment and/or prevention of a dystrophy affecting the corneal epithelium, the Bowman's layer, the corneal stroma, the Descemet's membrane and/or the corneal endothelium, preferably a posterior corneal dystrophy, more preferably FECD. The invention further relates to a method for the treatment of a disorder of the cornea, preferably a genetic disorder, said method comprising the steps of topically administering an ophthalmic composition according to the invention, allowing the entry of the corneal epithelium, the Bowman's layer, the corneal stroma, the Descemet's membrane and/or the corneal endothelium by the nucleic acid molecule, optionally allowing the entry of the cells within the corneal endothelium, and optionally allowing the passage of the nuclear membrane within the endothelium cells by the nucleic acid molecule.

In yet another embodiment, the invention relates to a method of treating a disorder of the cornea, preferably a hereditary corneal dystrophy, in a mammalian subject in need thereof, comprising the steps of providing an ophthalmic composition comprising: i) a nucleic acid molecule, ii) a viscosifying polymer, and iii) a solvent, wherein the nucleic acid molecule is at least partially complementary to a target (pre-) mRNA molecule causing the disorder, administering the ophthalmic composition topically to the anterior side of one or both corneas of the subject, allowing the entry of the nucleic acid molecule to a diseased cell within the corneal epithelium, the Bowman's layer, the corneal stroma, the Descemet's membrane and/or the corneal endothelium, and allowing the nucleic acid molecule to hybridize to a complementary sequence of a (pre-) mRNA molecule within the diseased cell. Preferably, the nucleic acid molecule modulates the splicing of the (pre-) mRNA, prevents or diminishes the formation of RNA foci, or the nucleic acid molecule causes a nuclease-dependent breakdown of the (pre-) mRNA. In a more preferred aspect, the invention relates to a method according to the invention, wherein the viscosifying polymer is selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine. Most preferred is a method according to the invention wherein the viscosifying polymer is HPMC.

### BRIEF DESCRIPTION OF THE DRAWINGS

FIG. 1 is a schematic representation of the frontal part of the eye, showing (enlarged on the left) from left to right: the corneal epithelium layer on the outside of the eye, the Bowman's layer between the epithelium and the stroma, the corneal stroma, the Descemet's membrane, the corneal endothelial layer and the anterior chamber fluid.

FIG. 2 shows the staining of mouse corneal layers, two days after topical administration of a composition comprising an AON (mQR421a), in this example targeted to the mouse USH2A pre-mRNA+Pluronic F127 to the corneal epithelium. Bright staining can only be observed above the corneal epithelium, not in the epithelium, Bowman's membrane, stroma or corneal endothelium.

FIG. 3 shows the staining of mouse corneal layers and retinas, two and fourteen days after topical application of a composition comprising mQR421a and another control (non-eye-related AON) in combination with hypromellose (HPMC) as a penetrating enhancer. Clear staining (and therefore presence) of the AONs is observed in the corneal endothelium (arrows). No staining was observed in the retinas.

FIG. 4 is a bar diagram showing a strong and significant decrease in normalized MALAT1 expression in the cornea after topical administration of a composition comprising i) a gapmer directed at MALAT1 transcripts and ii) hypromellose (HPMC) as a penetrating enhancer, in comparison to MALAT1 transcript expression after application of a negative control composition comprising a negative control gapmer and a control composition without any nucleic acid, both with hypromellose as a penetrating agent. MALAT1 expression was calculated by dividing the MALAT1 ddPCR signal by the GAPDH ddPCR signal. The reduction in normalized MALAT1 expression between the MALAT1 gapmer (mean of 0.12) and the no gapmer treated group (mean of 0.7) was significant with a p value of 0.0093, as is the reduction between the MALAT1 gapmer (mean of 0.12) group and the negative control gapmer (mean of 1.02) treated group (p value of 0.0168).

FIG. 5 is a bar diagram showing a strong and significant decrease in normalized MALAT1 expression in the cornea after topical administration and intravitreal administration of the MALAT1 specific gapmer and using an unrelated gapmer as a negative control. Shown are results obtained after topical administration of a single dose gapmer and sacrifice after 2 days (1 D,2 dS), a single dose gapmer and sacrifice after 5 days (1 D,5 dS), a dose gapmer followed by a second dose 2 days later and sacrifice 2 days after the second dose (2 D,2 dS), and results from mice that received a single dose intravitreally, and that were sacrificed 5 days later (IVT,5 dS).

FIG. 6 is a bar diagram showing no significant decrease in normalized MALAT1 expression in the retina after topical administration of the MALAT1 specific gapmer and the negative control gapmer. Significant downregulation could only be observed in the retina when the MALAT1 gapmer was administered intravitreally.

FIG. 7 is a bar diagram showing a strong and significant decrease in normalized MALAT1 expression in the cornea after topical administration of the MALAT1 specific gapmer in combination with a variety of HPMC concentrations (01.%, 0.2%, 0.3%, 0.4%, and 0.5%) and using an unrelated gapmer as a negative control (with 0.3% HPMC).

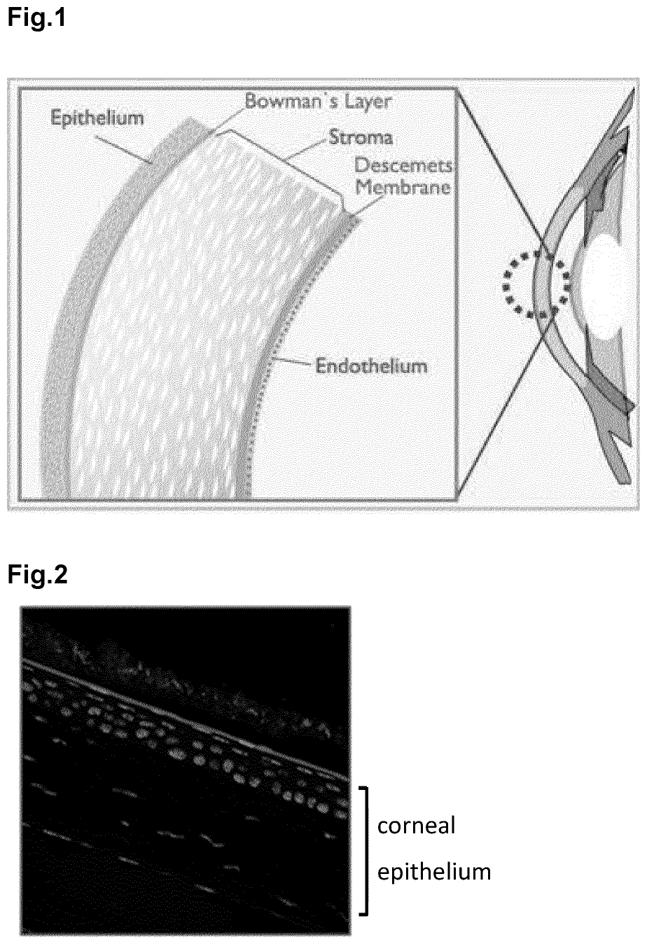

### DETAILED DESCRIPTION OF THE INVENTION

The present invention relates to methods and compositions that can be used in the prevention or treatment of genetic diseases, preferably those of the cornea, more preferably where the compositions do not significantly affect transcription and/or translation processes in the retina.

More in particular, the present invention relates to a composition comprising i) a nucleic acid molecule, ii) a penetration enhancer or viscosifying polymer, and iii) a solvent, for use in treating corneal diseases, wherein the nucleic acid molecule targets a pre-mRNA and/or mRNA from a gene comprising the genetic alteration causing the genetic disease. A preferred nucleic acid molecule is a single-stranded antisense oligonucleotide (AON) that is capable of interfering with splicing, and/or that may prevent the formation of RNA foci (preferably those that are caused by trinucleotide repeats (TNRs)). Another preferred nucleic acid molecule is also a single-stranded antisense oligonucleotide, but that (because of its RNA-DNA-RNA content) is generally referred to as a ‘gapmer’ having a two wings of RNA nucleotides and a gap of DNA nucleotides. A gapmer is preferably used to target (pre-) mRNA to downregulate expression of a protein from that (pre-) mRNA by causing the degradation of the mRNA once the gapmer has formed a double-stranded structure with that target RNA. A penetration enhancer that is used in a composition of the present invention is preferably a viscosifying polymer. More preferably, the penetration enhancer is a non-ionic viscosifying polymer. A particularly preferred non-ionic viscosifying polymer that is used in a composition of the present invention is hypromellose, short for hydroxypropyl methylcellulose (HPMC), a semisynthetic, inert, visoelastic polymer often used as eye drops, as well as an excipient and controlled-delivery component in oral medicaments. The inventors of the present invention have surprisingly found that the sole presence of hypromellose in the composition together with the nucleic acid, mixed together in a solvent, can cause the delivery of the nucleic acid molecule to the different layers within the cornea. To the best of the knowledge of the inventors, hypromellose has predominantly been used as a lubricant, for instance for the treatment of dry eyes, and not as a penetration enhancer. The inventors of the present invention have surprisingly found that when a nucleic acid molecule was combined with hypromellose and topically applied to the eye of mice, the nucleic acid molecule did pass the corneal epithelium, Bowman's layer and entered the cells of the corneal endothelium, whereas such could not be achieved by using Pluronic F127 (example section, below). Importantly, by using Pluronic F127, the nucleic acid molecule did not even reach the corneal epithelium.

Hypromellose is found in a variety of commercial products. As a food additive, hypromellose is an emulsifier, thickening and suspending agent, and an alternative to animal gelatin. It is non-toxic and its E number is E464. Hypromellose in an aqueous solution, like methylcellulose, exhibits a thermal gelation property. That is, when the solution heats up to a critical temperature, the solution congeals into a non-flowable but semi-flexible mass. Typically, this critical (congealing) temperature is inversely related to both the solution concentration of HPMC and the concentration of the methoxy group within the HPMC molecule, which in turn depends on both the degree of substitution of the methoxy group and the molar substitution. That is, the higher the concentration of the methoxy group, the lower the critical temperature. The inflexibility/viscosity of the resulting mass, however, is directly related to the concentration of the methoxy group (the higher the concentration, the more viscous or less flexible the resulting mass will be). Hypromellose augmentation as eye-drop material results in extended lubricant time presence on the cornea, which theoretically results in decreased eye irritation, especially in dry environments. On a molecular level, the polymer contains beta-linked D-glucose units that remain metabolically intact for days to weeks. On a manufacturing note, since hypromellose is a vegetarian substitute for gelatin, it is slightly more expensive to produce due to semisynthetic manufacturing processes. Hypromellose 2% solution has been documented to be used during surgery to aid in corneal protection and during orbital surgery. The inventors of the present invention questioned whether the polymer would also aid in the penetrance of the cornea and surprisingly found that indeed the presence of hypromellose could direct nucleic acid molecules across the epithelium, across the stroma and inner layers of the cornea to even reach the corneal endothelium, where the nucleic acid molecule (in the present case in the form of a single-stranded ribonucleotide and a gapmer), was able to enter the cells of the endothelium and exert an effect on transcript degradation (see the example section where a gapmer was used to target the long non-coding RNA MALAT1). This now opens a wide variety of applications that are alternatives for surgery or corneal replacement, in the treatment or prevention of genetic disorders affecting the different layers of the cornea beneath the tear film.

A preferred disease that is treated with the compositions of the present invention is Fuchs Endothelial Corneal Dystrophy (FECD), which is associated with the occurrence of excessive TNR expansions in intron 3 of the TCF4 transcript, causing excessive binding of the splice regulating protein MBNL1 to such excessive TNR expansions. FECD segregates into early-onset FECD and age-related FECD, which may be different diseases since guttae are not typically present in early-onset FECD. Early-onset FECD is rare and has been linked to genes such as Col82A2, encoding the α2-subunit of collagen VIII, a component of the endothelial basement membrane. In age-related FECD certain rare autosomal dominant mutations have been found in different genes, such as KCNJ13 (a potassium channel), SLC4A11 (a sodium-borate co-transporter) and ZEB1 (the Zinc-finger E-box homeodomain protein 1). Importantly however, the genetic basis of the majority of autosomal dominant age-related FECD has been attributed to the Transcription factor-4 (TCF4) gene following a genome-wide association study (Baratz K H et al. E2-2 protein and Fuch's corneal dystrophy. N Engl J Med 2010 363:1016-1024). In these studies, a Single-Nucleotide Polymorphism (SNP) was identified within an intron of the TCF4 gene: r5613872 on chromosome 18q21.2, which segregated specifically in age-related FECD patients. The increase in the risk of FECD development is calculated as a 30-fold increase in homozygous subjects and the r5613872 marker was able to discriminate between cases and controls with 76% accuracy. At least two regions of the TCF4 locus have been associated with development of FECD, following prior observations of FECD associating with a chromosomal region located at 18q21.2-18q21.32 (Sundin O H et al. A common locus for late onset Fuchs corneal dystrophy maps to 18q21.2-q21.32. Invest Ophthalmol Vis Sci 2006 47:3919-3926). Several other studies illustrated that the presence of a TCF4 trinucleotide repeat (TNR) was more predictive of FECD than the r5613872 marker (Wieben E D et al. A common trinucleotide repeat expansion within the transcription 4 (TCF4, E2-2) gene predicts Fuchs corneal dystrophy. PLoS One 2012 7:e49083; Wieben E D et al. Comprehensive assessment of genetic variants within TCF4 in Fuchs' endothelial corneal dystrophy. Invest Ophtalmol Vis Sci 2014 55:6101-6107; Mootha V V et al. Association and familial segregation of CTG18.1 trinucleotide expansion of TCF4 gene in Fuchs' endothelial corneal dystrophy. Invest Ophtalmol Vis Sci 2014 55:32-42; Stamler J F et al. Confirmation of the association between the TCF4 risk allele and Fuchs endothelial corneal dystrophy in patients from the Midwestern United States. Ophthalmic Genet. 2013 34(1-2):32-4; Kuot A et al. Association of TCF4 and CLU polymorphisms with Fuchs' endothelial dystrophy and implication of CLU and TGFBI proteins in the disease process. Eur J Hum Genet. 2012 20(6):632-8; Thalamuthu A et al. Association of TCF4 gene polymorphisms with Fuchs' corneal dystrophy in the Chinese. Invest Ophthalmol Vis Sci. 2011 52(8):5573-8; Xing C et al. Transethnic replication of association of CTG18.1 repeat expansion of TCF4 gene with Fuchs' corneal dystrophy in Chinese implies common causal variant. Invest Ophthalmol Vis Sci. 2014 55(11):7073-8; Nanda G G et al. Genetic association of TCF4 intronic polymorphisms, CTG18.1 and r517089887, with Fuchs' endothelial corneal dystrophy in an Indian population. Invest Ophthalmol Vis Sci. 2014 55(11):7674-80). FECD was observed to be associated with a (CTG)n TNR expansion in an intron region of the TCF4 gene that is different from the intron in which the r5613872 marker is located (Mootha et al. 2014; Wieben et al. 2012). It was shown that 79% of FECD patients (noted in leukocyte DNA) had 50 or more repeats (≥150 nucleotides), whereas 95% of case controls had repeat lengths of less than 40, which shows that a repeat length of 50 or more is highly predictive of FECD, whereas fewer repeats, between 40 and 50, also contribute to appearance of the disease. It is generally accepted in the field that the appearance of a TNR expansion at a size equal or greater than 40 repeats in the TCF4 gene is predictive of disease and indicative of a potential RNA toxicity mechanism leading to FECD (Du et al. RNA toxicity and missplicing in the common eye disease Fuch's endothelial corneal dystrophy. J Biol Chem 2015 290(10):5979-90). RNA foci were identified in fibroblasts from FECD patients that were both homozygous and heterozygous for TNR expansions in the TCF4 gene. No RNA foci were found in fibroblasts from unaffected individuals. Unaffected individuals generally appear to carry wild type TCF4 genes with around 20 TNRs. Heterozygote FECD patients (with fibroblasts wherein RNA foci were detected) carried one normal length allele (20 TNRs) and one allele with an expansion of ≥40 TNRs. In homozygote FECD patients both alleles contained 40 TNRs. Consequently, fewer than 40 repeats in the TCF4 expanded TNR regions can be considered a non-disease-causing genotype. RNA foci were also identified in the corneal endothelium of FECD patient samples, while none were found in unaffected individuals. The presence of such RNA foci appeared associated with a change in the RNA splicing patterns for several other genes (Du et al. 2015). The general conclusion is that the majority of FECD cases is caused by RNA toxicity in the corneal endothelial cells due to the presence of TNR expansions in intronic RNA derived from the TCF4 gene. RNA toxicity was found in patients that were either heterozygous or homozygous for the extended repeat and is likely the result of sequestration of proteins that interact with the RNA harboring the TNR expansions. Such proteins—through this sequestration—can no longer perform their normal function in the cells.

In a preferred aspect, the present invention relates to compositions according to the invention for use in the prevention or treatment of FECD, by delivering to the corneal endothelium AONs that bind to the excessive TNR expansion in the transcripts of the TCF4 gene, thereby preventing the unwanted binding of proteins to the excessive TNR expansion. A hallmark of MBNL1 binding to excessive TNR expansions is the formation of so-called RNA foci in the nucleus of diseased cells of a patient. Hence, the compositions of the present invention are preferably used to treat or prevent genetic (eye) diseases such as FECD, by removal or preventing the formation of RNA foci, particularly in corneal endothelial cells.

DM1 is, like FECD, a disease resulting from RNA toxicity, and EP2049664B1 discloses methods for treating DM1 using AONs targeting TNR expansions in transcripts of the human DMPK gene. EP2049664B1 discloses AONs having the sequence 5′-(CAG)n-3′ to treat a variety of human cis-element repeat instability associated disorders, such as HD, spinocerebellar ataxia, Haw River syndrome, X-linked spinal and bulbar muscular atrophy and dentatorubral pallidoluysian atrophy, DM1, spinocerebellar ataxia type 8, and Huntington's disease type 2. The TNR repeat expansions in DM1 are found in the 3′-UTR of the DMPK gene. Others describe the use of (CAG)₇ AONs to target transcripts of exon 15 of the human DM PK gene correlated with DM1 (Mulders et al. 2009. Triplet-repeat oligonucleotide-mediated reversal of RNA toxicity in myotonic dystrophy. Proc Natl Acad Sci USA 106(33):13915-20). The authors assume that both the cytoplasmic pool of mRNA, as well as the nuclear pool of primary and mature expanded (CUG)n transcripts served as targets. DM1 is an RNA-toxicity mediated disease and toxic DMPK pre-mRNAs contain expanded TNRs with the same repeating unit (CUG) as the TCF4 transcript in FECD patients. It may seem that MBNL1 is sequestered in DM1 patients in a similar fashion as in FECD patients.

Interestingly, if sequestration of MBNL1 or other factors common to these RNA foci are mechanistically involved in pathology of FECD, then DMPK containing TNR expansions should convey a similar risk of developing FECD if DMPK is found to be expressed in the corneal endothelium. Interestingly, DMPK is in fact expressed in the human eye (Winchester C L et al. Characterization of the expression of DMPK and SIX5 in the human eye and implications of pathogenesis in myotonic dystrophy. Hum Mol Genet. 1999 8:481-492) and FECD has been found in myotonic dystrophy patients: In a cohort of four DM patients each patient had bilateral FECD (Gattey D et al. Fuchs endothelial corneal dystrophy in patients with myotonic dystrophy: a case series. Cornea 2014 33:96-98). Also, it has been noted in post-mortem eyes from myotonic dystrophy patients that there is considerable loss of corneal endothelial cells which is a clinical hallmark of advanced FECD (Winchester et al. 1999). DM1 patients who develop FECD can be treated for FECD using an AON approach. By targeting TCF4 transcripts with excessive TNR expansions in intron 3, as is the case in FECD patients, such patients can effectively be treated for FECD, using the compositions of the present invention.

The present invention relates to an ophthalmic composition for the treatment and/or prevention of a disorder of the cornea, said composition comprising: i) a nucleic acid molecule, ii) a viscosifying polymer, and iii) a solvent, wherein the nucleic acid molecule is at least partially complementary to, and capable of binding a target (pre-) mRNA molecule, and wherein the viscosifying polymer enables the nucleic acid molecule to penetrate through the layers within the cornea after topical administration of the composition. Preferably, the nucleic acid molecule and viscosifying polymer are mixed with the solvent, preferably to form a homogeneous mixture. The solvent may be any suitable pharmaceutically acceptable solvent known to the person skilled in the art. One example of a suitable solvent is PBS. In a preferred embodiment, the composition does not affect transcriptional and/or translation processes in the retina. Many corneal diseases exist, wherein the retina is healthy. Hence, it is preferred that unwanted side effects in the retina do not occur when the composition is administered topically. That the cornea can be targeted using the compositions of the invention, without affecting the retina is shown in the accompanying examples. The skilled person is able to determine relatively easy whether a nucleic acid molecule, by following the teaching herein, did or did not reach any of the different layers of the cornea after topical administration, wherein topical administration means an administration of the composition on top of the corneal epithelium and mixes with the tear film. Even though the tear film is constantly renewed, the composition of the present invention is such that it allows enough time for the nucleic acid molecule to penetrate the epithelium, and the other layers posterior of it. By using the composition of the present invention there is no need for sustained delivery devices such as disclosed in WO2014/025792, where drug delivery is achieved through nanowafers or microwafers made from biodegradable material in which reservoirs contain the drug of interest (not mixed with the biodegradable material). With the present invention, there is no strong need for sustained release as the penetrating agent allows the entry of the nucleic acid through the corneal layers after topical administration of the composition. The composition of the present invention is preferably applied as a homogeneous mixture at the topical side of the cornea, for instance as drops or as a film. Before the drops or film is washed out, the penetrating agent has enabled the entry of the nucleic acid into the corneal layers beneath the epithelium. Moreover, by using the composition of the present invention, the entire surface of the cornea can be covered and treated, which is beneficial in the case of diseases that affect the entire layer within the cornea. Any disorder that can be treated with an antisense single-stranded oligonucleotide can in principle be treated (or prevented, or diminished, or ameliorated) by using the teaching of the present invention. The examples show the use of hypromellose, but the skilled person will understand that even though hypromellose is generally used to treat topical events such as dry eye, other viscosifying polymers or lubricants that are like hypromellose can be applied in a composition of the present invention, and their penetrating efficiency can be determined based on the current teaching. Preferably, the viscosifying polymer is selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine. The best results are achieved with HPMC, which is the most preferred viscosifying polymer for the compositions of the present invention. As shown in the accompanying examples, different percentages of HPMC could be used to achieve the same result in nucleic acid entry and subsequent activity (targeting a IncRNA to downregulate its expression). A preferred range in which the HPMC is used in a composition of the present invention is from about 0.1% to about 0.5%, more preferably about 0.3%. Preferably, the disorder is a genetic disorder, such as a hereditary corneal dystrophy, and wherein the target (pre-) mRNA molecule is the cause of the genetic disorder. Antisense oligonucleotides often are used to target (pre-) mRNA that is the cause of the genetic disorder but may also be used to target RNA molecules for diminishing immune-related proteins that indirectly are involved in the occurrence of disease. Preferably, the oligonucleotides that are used in the composition of the present invention are suitable for manufacturing, but also for entry into cells and passage of the nuclear membrane if the target RNA molecule is present in the nucleus. Hence, the nucleic acid molecule preferably comprises 10 to 50, more preferably 10 to 40, even more preferably 10 to 30 contiguous nucleotides, and more in particular preferably 10, 11, 12, 13, 14, 15, 16, 17, 18, 19, 20, 21, 22, 23, 24, 25, 26, 27, 28, 29, or 30 contiguous nucleotides. It is preferred that a single nucleic acid molecule (with the complementary contiguous nucleotides) is used in a medicament, but a medicament may also comprise different kinds of nucleic acid molecules. The complementarity is preferably 100%, but one or two nucleotides may differ in complementarity in respect of the target RNA molecule, for instance, when the nucleic acid molecule is complementary to the wild type sequence, while the target RNA molecule comprises a mutation in the region of complementarity. However, 100% complementarity is preferred. In a preferred embodiment, the nucleic acid molecule is a single-stranded antisense oligonucleotide (AON) that modulates splicing of the target pre-mRNA; or that prevents or reduces RNA toxicity as outlined herein for DM1 and FECD. In another preferred embodiment, the nucleic acid molecule is a gapmer that downregulates expression of the target (pre-) mRNA. A gapmer generally comprises a middle section built of DNA nucleotides, whereas the two wing segments are generally RNA nucleotides. In a preferred aspect, the nucleic acid molecule is non-naturally chemically modified to render it more stable towards nuclease breakdown and/or to increase its affinity towards the target (pre-) mRNA molecule. Potential chemical modifications are well known to the person skilled in the art, and preferred chemical modifications may depend on the target RNA molecule, the length of the oligonucleotide and the strength of hybridizing. Such experimentation, to explore the different chemical moieties for the best effect within the cells of the corneal tissues, is well within the general skill of the artisan. In a preferred embodiment, the disorder that is treated with a composition according to the present invention is a dystrophy affecting the corneal epithelium, the Bowman's layer, the corneal stroma, the Descemet's membrane and/or the corneal endothelium. The different hereditary corneal dystrophies that may be treated and/or prevented by the teaching of the present invention are all hereditary corneal dystrophies of the 4 categories outlined above. Preferably, the disorder that is treated and/or prevented is a posterior corneal dystrophy, preferably Fuchs' Endothelial Corneal Dystrophy (FECD).

In yet another embodiment, the invention relates to an ophthalmic composition according to the invention, for use in the treatment and/or prevention of a dystrophy affecting the corneal epithelium, the Bowman's layer, the corneal stroma, the Descemet's membrane and/or the corneal endothelium, preferably a posterior corneal dystrophy, more preferably FECD.

In yet another embodiment, the invention relates to the use of a nucleic acid molecule and a viscosifying polymer, as outlined herein, in the manufacture of a medicament for the prevention or treatment of a dystrophy affecting the corneal epithelium, the Bowman's layer, the corneal stroma, the Descemet's membrane and/or the corneal endothelium, preferably a posterior corneal dystrophy, more preferably FECD. In a preferred aspect, the viscosifying polymer is selected from the group consisting of: hydroxypropyl methylcellulose (hypromellose, or HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine.

In yet another embodiment, the invention relates to a method for the treatment of a disorder of the cornea, preferably a genetic disorder, said method comprising the steps of topically administering an ophthalmic composition according to the invention, allowing the entry of the corneal epithelium, the Bowman's layer, the corneal stroma, the Descemet's membrane and/or the corneal endothelium by the nucleic acid molecule, optionally allowing the entry of the cells within the corneal endothelium, and optionally allowing the passage of the nuclear membrane within the endothelium cells by the nucleic acid molecule. Preferably, the genetic disorder is a corneal disorder of a mammal, more preferably of a human subject.

In yet another embodiment, the invention relates to a method of treating a disorder of the cornea, preferably a hereditary corneal dystrophy, in a mammalian subject in need thereof, comprising the steps of providing an ophthalmic composition comprising: i) a nucleic acid molecule, ii) a viscosifying polymer, and iii) a solvent, wherein the nucleic acid molecule is at least partially complementary to a target (pre-) mRNA molecule causing the disorder, administering the ophthalmic composition topically to the anterior side of one or both corneas of the subject, allowing the entry of the nucleic acid molecule to a diseased cell within the corneal epithelium, the Bowman's layer, the corneal stroma, the Descemet's membrane and/or the corneal endothelium; and allowing the nucleic acid molecule to hybridize to a complementary sequence of a (pre-) mRNA molecule within the diseased cell. In a preferred aspect, the nucleic acid molecule modulates the splicing of the (pre-) mRNA, prevents or diminishes the formation of RNA foci, or wherein the nucleic acid molecule causes a nuclease-dependent breakdown of the (pre-) mRNA. Preferably, the invention relates to a method according to the invention, wherein the viscosifying polymer is selected from the group consisting of: hydroxypropyl methylcellulose (HPMC, or hypromellose), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine.

In yet another aspect the invention relates to a use of an ophthalmic composition for topical administration to the eye of a mammalian subject suffering from a disorder of the cornea, preferably a hereditary corneal dystrophy, wherein the composition comprises: i) a nucleic acid molecule, ii) a viscosifying polymer, and iii) a solvent, wherein the nucleic acid molecule is at least partially complementary to a target (pre-) mRNA molecule causing the disorder, and wherein the viscosifying polymer enables the nucleic acid molecule to penetrate through the different layers of the cornea.

It is preferred that a single-stranded antisense nucleic acid molecule present in a composition of the invention comprises one or more residues that are modified to increase nuclease resistance (e.g. in the case of splice modulation), and/or to increase the affinity of the nucleic acid molecule for the target sequence. Therefore, in a preferred embodiment, the nucleic acid molecule comprises at least one nucleotide analogue or equivalent, wherein a nucleotide analogue or equivalent is defined as a residue having a modified base, and/or a modified backbone, and/or a non-natural internucleoside linkage, or a combination of these modifications. In a preferred embodiment, the nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells.

According to one embodiment of the invention the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages. In accordance with this embodiment, a preferred nucleotide analogue or equivalent comprises a Peptide Nucleic Acid (PNA), having a modified polyamide backbone (Nielsen et al. 1991 Science 254:1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar 2005 Chem Commun 495-497). Since the backbone of a PNA molecule contains no charged phosphate groups, PNA-RNA hybrids are usually more stable than RNA-RNA or RNA-DNA hybrids, respectively (Egholm et al. 1993 Nature 365:566-568).

According to another embodiment of the invention, the backbone comprises a morpholino nucleotide analog or equivalent, in which the ribose or deoxyribose sugar is replaced by a 6-membered morpholino ring. A most preferred nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

In yet a further embodiment, a nucleotide analogue or equivalent comprises a substitution of one of the non-bridging oxygens in the phosphodiester linkage. This modification slightly destabilizes base pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

A further preferred nucleotide analogue or equivalent comprises one or more sugar moieties that are mono- or di-substituted at the 2′, 3′ and/or 5′ position such as a —OH; -F; substituted or unsubstituted, linear or branched lower (C1-C10) alkyl, alkenyl, alkynyl, alkanyl, allyl, or aralkyl, that may be interrupted by one or more heteroatoms; O—, S—, or N-alkyl; O—, S—, or N-alkenyl; O—, S— or N-alkynyl; O—, S—, or N-allyl; O-alkyl-O-alkyl, -methoxy, -aminopropoxy; methoxyethoxy; -dimethylaminooxyethoxy; and -dimethylaminoethoxyethoxy. The sugar moiety can be a furanose or derivative thereof, or a deoxyfuranose or derivative thereof, preferably ribose or derivative thereof, or deoxyribose or derivative thereof. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-O,4′-C-ethylene-bridged nucleic acid (Morita et al. 2001 Nucleic Acid Res Supplement No. 1: 241-242). These substitutions render the nucleotide analogue or equivalent RNase H and nuclease resistant and increase the affinity for the target RNA.

It is understood by a skilled person that it is not necessary for all internucleosidic linkages in an antisense oligonucleotide to be modified. For example, some internucleosidic linkages may be unmodified, whereas other internucleosidic linkages are modified. AONs comprising a backbone consisting of one form of (modified) internucleosidic linkages, multiple forms of (modified) internucleosidic linkages, uniformly or non-uniformly distributed along the length of the AON are all encompassed by the present invention. In addition, any modality of backbone modification (uniform, non-uniform, mono-form or pluriform and all permutations thereof) may be combined with any form or of sugar or nucleoside modifications or analogues mentioned below. An especially preferred backbone for the AONs according to the invention is a uniform (all) phosphorothioate (PS) backbone.

In another embodiment, a nucleotide analogue or equivalent of the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

It is understood by a skilled person that it is not necessary for all positions in an antisense oligonucleotide to be modified uniformly. In addition, more than one of the aforementioned analogues or equivalents may be incorporated in a single antisense oligonucleotide or even at a single position within an antisense oligonucleotide. In certain embodiments, an antisense oligonucleotide of the invention has at least two different types of analogues or equivalents. According to another embodiment oligonucleotides in an ophthalmic composition according to the invention comprise a 2′-O (preferably lower) alkyl phosphorothioate antisense oligonucleotide, such as 2′-0-methyl modified ribose (RNA), 2′-O-methoxyethyl modified ribose, 2′-0-ethyl modified ribose, 2′-O-propyl modified ribose, and/or substituted derivatives of these modifications such as halogenated derivatives.

An effective and especially preferred oligonucleotide format comprises 2′-O-methyl modified ribose moieties with a phosphorothioate backbone, preferably wherein substantially all ribose moieties are 2′-O-methyl modified and substantially all internucleosidic linkages are phosphorothioate linkages. Another effective and especially preferred oligonucleotide format comprises 2′-O-methoxyethyl (2′-MOE) modified ribose moieties with a phosphorothioate backbone, preferably wherein substantially all ribose moieties are 2′-O-methoxyethyl modified and substantially all internucleosidic linkages are phosphorothioate linkages.

A particularly preferred ophthalmic composition according to the invention comprises an oligonucleotide that contains a sequence complementary to (part of) a TNR expansion and comprises a multitude of a sequence that is complementary to the triplet sequence in the expansion. Although a TNR may be referred to as a repeating sequence of CUG triplets (from 5′ to 3′), the nature of DNA (and corresponding RNA) makes that such can also be written as UGC repeats or as GCU repeats, depending on what is considered to be the first nucleotide of the triplet. This means that the antisense oligonucleotides may either start with any of the nucleotides that is complementary to one of the nucleotides in the triplet: CAG, GCA, or AGC (from 5′ to 3′). Targeting 5′-(CUG)n-3′ TNRs preferably takes place by using AONs with complementary sequences formed through canonical Watson-Crick base-pairs: 5′-(CAG)m-3′, or through wobble base-pairs, such as 5′-(CAI)m-3′, 5′-(CGG)m-3′, 5′-(CGI)m-3′, 5′-(CIG)m-3′, 5′-(CII)m-3′, 5′-(UAG)m-3′, 5′-(UAI)m-3′, 5′-(UGG)m-3′, 5′-(UGI)m-3′, 5′-(UIG)m-3′ and 5′-(UII)m-3′. When the second nucleotide shifts one position towards the 5′ end and becomes the first nucleotide of the repeat, the following repeated sequences are then contemplated: 5′-(AGC)m-3′, 5′-(AIC)m-3′, 5′-(GGC)m-3′, 5′-(GIC)m-3′, 5′-(IGC)m-3′, 5′-(IIC)m-3′, 5′-(AGU)m-3′, 5′-(AIU)m-3′, 5′-(GGU)m-3′, 5′-(GIU)m-3′, 5′-(IGU)m-3′ and 5′-(IIU)m-3′, When the third nucleotide takes the place of the first nucleotide then the following repeated sequences are contemplated: 5′-(GCA)m-3′, 5′-(ICA)m-3′, 5′-(GCG)m-3′, 5′-(ICG)m-3′, 5′-(GCI)m-3′, 5′-(ICI)m-3′, 5′-(GUA)m-3′, 5′-(IUA)m-3′, 5′-(GUG)m-3′, 5′-(IUG)m-3′, 5′-(GUI)m-3′ and 5′-(IUI)m-3′. Clearly, in a CUG sequence that is repeated 40 or more times, one can also recognize UGC repeats and GCU repeats.

The current invention makes use of a nucleic acid molecule that is preferably an AON that is capable of binding a trinucleotide repeat (TNR) expansion that comprises the sequence 5′-(CUG)n-3′, wherein n is long enough to cause RNA toxicity in a cell in which such TNR expansions are transcribed. The TCF4 gene is such a gene that, when comprising 40 or more of the TNRs, causes FECD by sequestration of normal cellular proteins to the TNR expansions in the cells of the corneal endothelium.

Although the invention is exemplified using ‘naked’ or ‘gymnotic’ AONs as molecules that are capable of binding to a TNR expansion, persons having ordinary skill in the art will recognize that other molecules that are capable of binding to nucleic acid sequences, more in particular TNRs, yet more particularly 5′-(CUG)n-3′ expansions are encompassed by the invention. Examples of such molecules are proteins, such as Zinc-Finger proteins, antibodies or antibody fragments, aptamers, bivalent ligands (Haghighat Jahromi A et al. Developing bivalent ligands to target CUG triplet repeats, the causative agent of Myotonic Dystrophy Type 1. J Med Chem 2013. 56:9471-9481) and the like.

The invention thus provides ophthalmic compositions for use in a method for the prevention and/or treatment of an unstable cis-element DNA repeat associated genetic disorder, preferably an eye dystrophy, more preferably FECD. The invention relates to a method comprising the steps of topically administering an ophthalmic composition according to the invention and allowing the nucleic acid molecule within the composition to pass the different layers of the cornea, and allowing the entry of the nucleic acid molecule into the cells of the cornea tissues within the cornea, such as the cells of the endothelium. Preferably, the method comprises the step of allowing the nucleic acid molecule to pass the nuclear membrane to exert its effect in the nucleus, preferably by targeting the (pre-) mRNA within that nucleus.

The oligonucleotides that are the nucleic acid molecules within the ophthalmic compositions of the present invention are preferably single stranded, chemically modified and synthetically produced. An nucleic acid molecule present in a composition according to the invention may be from 8 to 200 nucleotides in length, preferably between 10 and 100, more preferably between 10 and 50, even more preferably comprises 10 to 40 consecutive (or contiguous) nucleotides, and most preferably comprises 10, 11, 12, 13, 14, 15, 16, 17, 18, 19, 20, 21, 22, 23, 24, 25, 26, 27, 28, 29, or 30 consecutive nucleotides. The oligonucleotides, when targeting a TNR preferably comprises between 2 and 66 repetitive units consisting of 3 nucleotides, preferably 5, 6, 7, 8, 9, 10, 11, 12, 13, 14, 15, 16 or 17 of such repetitive units. A TNR comprises at least two repetitive units of identical repeating units of three nucleotides (=trinucleotide), which means that when a trinucleotide is repeated 3 times, the length of the TNR is at least 9 nucleotides and that when a trinucleotide is repeated 7 times, the length of the TNR is at least 21 nucleotides. The length of the TNR that causes disease is dependent of the disease, which, in the case of FECD is equal to or more than 40 repeats of the repeating unit, usually more than 45, even more usually more than 50, although this may differ from patient to patient. It should be clear that the AONs of the present invention are complementary to several repetitive units (a minimum of two) in the target RNA, but this does not mean the AONs have to consist of a multiple of 3 nucleotides. For example, the AON may comprise, in addition to a central portion that is complementary to two or more repeating units, 1 or 2 complementary nucleotides at one end (5′ or 3′) or both ends (5′ and 3′) of the AON. In other words, an AON of the invention may be entirely complementary to a sequence within a TNR expansion in a target RNA, while the complementary region of the AON does not consist of a multiple of 3. An oligonucleotide may be 9 nucleotides in length, yet comprise only 2 repetitive CAG units, for instance.

Non vectored AONs for uses as contemplated herein are typically to be used in dosages ranging from 0.0001 to 200 mg/kg, preferably from 0.001 to 100 mg/kg, more preferably from 0.01 to 50 mg/kg, depending on the disease, the target organ or tissue, and the route of administration. For eye diseases, such as FECD a suitable dosage is upon topical administration would be between 0.05 mg and 5 mg, preferably between about 5.0 μg and 1.0 mg per eye, such as about 5.0 μg, 10 μg, 50 μg, 100 μg, 150 μg, 200 μg, 250 μg, 300 μg, 400 μg, 500 μg, 600 μg, 700 μg, 800 μg, 900 μg, or 1000 μg per eye. Dosing may be daily, weekly, monthly, quarterly, once per year, depending on the route of administration and the need of the patient.

It will be clear to a person having ordinary skill in the art to which this invention pertains, that the details of treatment will need to be established in accordance with and depending on such factors as the sequence and chemistry of the oligonucleotide(s), the route of administration, the formulation, the dose, the dosing regimen, the format (vector or non-vectored AON), the age and weight of the patient, the stage of the disease and so forth, which may require further non-clinical and clinical investigation.

### EXAMPLES

### Example 1. Delivery of Single-Stranded Antisense Oligonucleotides (AONs) to the Corneal Endothelium Through Topical Administration

Usher syndrome (USH, or just ‘Usher’) and non-syndromic retinitis pigmentosa (NSRP) are degenerative diseases of the retina. WO 2016/005514, WO 2017/186739 and WO 2018/055134 disclose AONs for the treatment of Usher by targeting retinal cells for splice modulation of the USH2A pre-mRNA (exon 13 and exon 50 skipping, as well as skipping of pseudo exon 40, or PE40, form the USH2A pre-mRNA). Although the AONs are meant to target the retina, one of the AONs, also referred to as QR-421a (or in fact its mouse equivalent mQR421a, see WO2018/055134) was used by the inventors of the present invention in an initial trial experiment to see whether it could be traced back in all layers of the cornea after topical administration (applying it to the corneal epithelium) on the mouse eye. mQR421a would serve as a control nucleic acid. For this, 200 μg mQR421a (40 μg/μl) was locally administered to the cornea of C57BL wild type mice (under isoflurane anesthesia for 15 min) by direct lubrication in a 15% Pluronic F127 (Sigma Aldrich) formulation, pH 7.4. Two days after administration, the mice were sacrificed, and their corneas were isolated and stained. The results are shown in FIG. 2. The AON was visualized (in red) by Fluorescence In Situ Hybridization (FISH). In blue the nuclei are stained with Hoechst 33342. It was concluded that mQR421a could not be found in any of the corneal layers and got stuck outside de corneal epithelium.

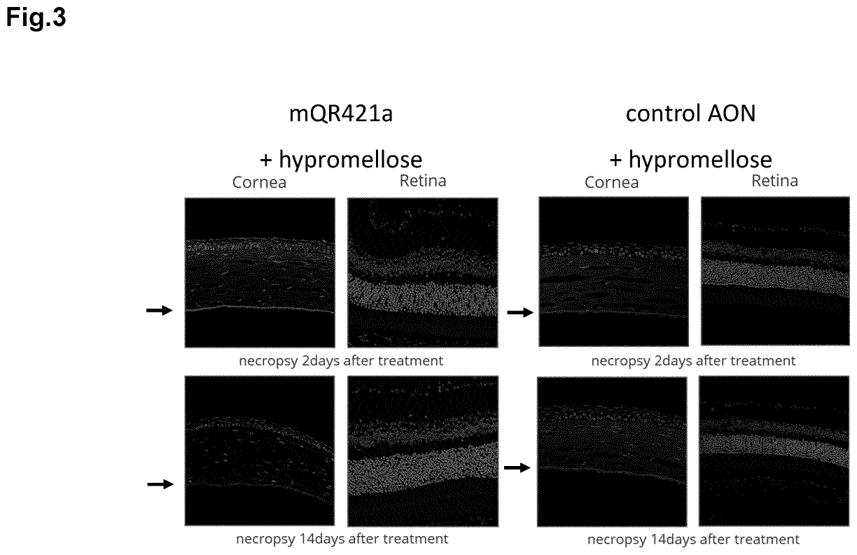

In a next experiment, mQR421a and another control AON (not related to an eye disease) were formulated in a 0.3% (w/w) hypromellose (HPMC) (Sigma Aldrich), prepared in PBS pH 7.4 at a concentration of 40 μg/μl AON. In total 200 μg AON was administered in 5 μl HPMC gel to the cornea of C57BL wild type mice while under isoflurane anesthesia for 15 min. Mice were sacrificed 2 and 14 days after dosing using CO₂ asphyxiation. Their corneas and retinas were isolated and snap-frozen in liquid nitrogen and stored at −80° C. prior to staining, which was performed as described above for mQR421a (the other AON was linked to a Cy5 label that stained by itself, and no FISH was used for this oligonucleotide). Results are shown in FIG. 3. In contrast to what was observed when using Pluronic F127, now both mQR421a and the control AON could be detected in the corneal endothelium 2 days and 14 days upon treatment. Using FISH, mQR421a was visualized in all layers of the cornea, even after 14 days of treatment, whereas the control AON was detectable in the stroma and corneal endothelium. AON delivery was local to the cornea as no AON was detectable in the retina. This now shows that the inventors were able to deliver nucleic acid molecules, in this case AONs, to all cells of the different layers of the cornea, by using hypromellose as the viscosifying polymer, after topical administration on the corneal epithelium.

### Example 2. Functional Downregulation of Transcript Expression in Corneal Endothelium After Topical Application of a Gapmer, Using Hypromellose as a Penetrating Enhancer

The inventors then questioned whether it was possible to show a functional effect of applying a nucleic acid molecule through topical administration (onto the corneal epithelium, using hypromellose) and delivering the nucleic acid molecule to all the layers of the cornea. For this, a certain type of oligonucleotide was chosen, generally referred to as a gapmer, which is a single-stranded antisense oligonucleotide molecule generally containing two wings segments comprising RNA nucleosides and a center part that is made of DNA. The gapmer hybridizes to its target sequence and causes a nuclease breakdown of the resulting double-stranded complex, thereby downregulating the expression of the target RNA. As a target, the inventors selected Metastasis Associated Lung Carcinoma Adenocarcinoma Transcript 1 (MALAT1), which serves here as an example only. MALAT1, also known as NEAT1, is a large, infrequently spliced non-coding RNA (IncRNA) that is highly conserved amongst mammals and highly expressed in the nucleus and cytoplasm.

A mouse-specific MALAT1 gapmer (5′-GTC ACA ATG CAT TCT A-3′; SEQ ID NO: 1) was tested in a single dose experiment with a treatment duration of 2 days on C57BL wild type mice. The gapmer was formulated in 0.3% (w/w) HPMC (Sigma Aldrich) prepared in PBS pH7.4 (Gibco). In total 150 μg gapmer was administered to the cornea by direct lubrication of 5 μl HPMC gel containing 30 μg/μl gapmer while mice were under isoflurane anesthesia for 15 min, like the experiment described above. Both the left and right eye of three mice were treated (six eyes in total). Control mice received a negative control gapmer that does not hybridize to MALAT1 RNA (5′-GCT CCC TTC AAT CCA A-3; SEQ ID NO: 2), which was used in the same way. A control group received 0.3% HPMC gel without added nucleic acid molecules.

Two days after treatment, mice were sacrificed using CO₂ asphyxiation. The corneas were isolated and snap-frozen in liquid nitrogen and stored at −80° C. prior to RNA isolation that was performed as follows. The beads of a quarter-filled filled green bead magnalyzer tube (Roche) were transferred to a single cornea, to which 350 μl RLT Plus buffer from the RNeasy Plus Mini Kit (Qiagen) was added. The cornea tissue was immediately homogenized using the magnalyzer at 5000 rpm for 30 sec. After a cooling period of 1 min on ice, tissues were homogenized again using the magnalyzer at 5000 rpm for 30 sec. Samples were centrifuged for 3 min at 4° C. The supernatant was transferred to a gDNA Eliminator spin column placed in a 2 ml collection tube from the RNeasy Plus Mini Kit (Qiagen) and centrifuged for 30 sec. An equal volume of 70% ethanol was added to the flow-through and mixed well by pipetting. The sample was transferred to an RNeasy spin column (RNeasy Plus Mini Kit, Qiagen) placed in a 2 ml collection tube and centrifuged for 30 sec. Flow-through was discarded. 700 μl Buffer RW1 (RNeasy Plus Mini Kit, Qiagen) was added to the RNeasy spin column (in a 2 ml collection tube) and centrifuged for 30 sec. Flow-through was discarded. 500 μl Buffer RPE (RNeasy Plus Mini Kit, Qiagen) was added to the RNeasy spin column (in a 2 ml collection tube) and centrifuged for 30 sec. Flow-through was discarded and the RNeasy spin column was placed in a new 2 ml collection tube (RNeasy Plus Mini Kit, Qiagen). 500 μl Buffer RPE (RNeasy Plus Mini Kit, Qiagen) was added to the RNeasy spin column (in a 2 ml collection tube) and centrifuged for 2 min at 10.000×g followed by a centrifugation of 1 min at full speed. The RNeasy spin column was placed in an RNase-free 1.5 ml Eppendorf tube and 15 μl RNase free water (RNeasy Plus Mini Kit, Qiagen) was added to the RNeasy spin column. The RNeasy spin column was centrifuged for 1 min to eluate the RNA. RNA concentration was determined using NanoDrop (Thermoscientific) and stored at −80° C. until further use.

MALAT1 transcripts were detected in a technical duplicate using a TaqMan assay (Applied Biosystems (FAM)) in a one-step reverse transcript digital droplet PCR (one-step RT-ddPCR) assay. To normalize MALAT1 transcripts, GAPDH transcripts were analyzed in a technical duplicate by one-step RT-ddPCR using a TaqMan assay (Applied Biosystems (FAM)). Table 1 shows the PCR protocol used for both the MALAT1 and the GAPDH assay. All RNA samples were diluted to a working concentration of 0.00375 ng/μl using nuclease free water (Ambion). A one-step RT-ddPCR was performed in technical duplicate using 0.015 ng RNA sample and the one-step RT-ddPCR advanced kit for Probes (BioRad). Transcript levels (total copies) were measured according to the manufacture's recommendations. The QX200 Droplet generator was used to generate droplets (BioRad) and PCR was run on a T100 Thermo Cycler (BioRad). Upon PCR, droplets were analyzed on the QX200 droplet reader (BioRad). Data analysis was performed using the Quantasoft software (BioRad). Only samples in which more than 8000 droplets were detected were included in the data analysis. Water and minus RT samples were included as negative controls. The water samples were used to determine the lower limit of detection for the MALAT1 and GAPDH assay. Any sample below the lower limit of detection was omitted from further data analysis. MALAT1 transcript levels were normalized to GAPDH transcript levels by calculating the MALAT1/GAPDH ratio using the average droplet vales of the technical duplicates.

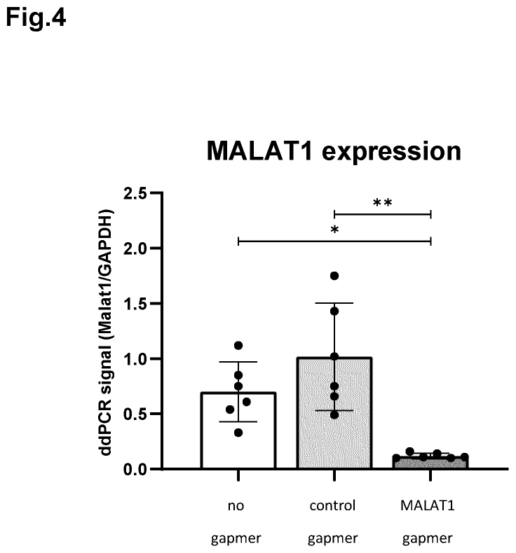

A Brown-Forsythe and Welsh ANOVA test was performed on the relative MALAT1 ddPCR signals because the standard deviations of the different treatment groups (MALAT1 gapmer—negative control gapmer—no AON) were not equal. Results, given in FIG. 4, show a significant reduction in MALAT1 expression levels in the MALAT1 gapmer treated group compared to the negative control gapmer treated group (p=0.0168), as well as compared to the no gapmer treated group (p=0.0093). The residual MALAT1 expression level in the MALAT1 gapmer treated group was 12% compared to the negative control gapmer treated group and 17% compared to the no gapmer treated group. It was found that knockdown efficiencies were very high: 88% (compared to negative control gapmer treated group) and 83% (compared to no gapmer treated group), which shows that the inventors were able to obtain a significant functional effect of downregulating transcript expression in the corneal endothelium layer, after applying (topically, on the outer surface of the eye) a composition comprising a nucleic acid molecule (here a gapmer) and a penetrating enhancer (here hypromellose).

TABLE 1
PCR program settings for one-step RT-ddPCR advanced
kit for probes on the T100 Thermo cycler.

| PCR Program | Temp ° c. | Time | | Cycles |
|---|---|---|---|---|
| Reverse transcription | 43.5 | 1 | h | 1 |
| Enzym activation | 95 | 10 | min | 1 |
| Denaturation | 95 | 30 | sec | 40 |
| Annealing/Extension | 60 | 1 | min | |
| Enzyme deactivation | 98 | 10 | min | 1 |

| | | | |
|---|---|---|---|
| Hold | 12 | ∞ | 1 |

Ramp rate settings: 2-3 degrees/sec

### Example 3. Effect on the Retina After Topical Application of a Nucleic Acid Molecule

The inventors wondered whether the functional effect of applying a nucleic acid molecule through topical administration (onto the corneal epithelium, using hypromellose) as described above would be limited to the corneal layers or whether the retina would also be reached. In the case of corneal endothelial disorders, it is preferred that the healthy retina is not reached, where it may potentially cause unwanted side-effects. Like what has been described above, the investigators selected the gapmer that targets MALAT1, which serves here as an example only. MALAT1, also known as NEAT1 is a large, infrequently spliced non-coding RNA (IncRNA) that is highly conserved amongst mammals and highly expressed in the nucleus.

The mouse specific MALAT1 gapmer (SEQ ID NO: 1) was formulated and administered as described above. Both the left and right eye of three mice were treated (six eyes in total). Three treatment groups were dosed with a MALAT1 gapmer containing 0.3% (w/w) HPMC gel. The first group received one dose and was sacrificed 2 days after (1 D,2 dS). The second group received one dose and was sacrificed 5 days after (1 D,5 dS). The third group received an initial dose, followed by a second dose 2 days after the first dose and was sacrificed 2 days after the last dose (2 D,2 dS). For each treatment group control mice received the negative control gapmer (SEQ ID NO: 2), which was used in the same way. The study further included mice that were injected intravitreally (IVT). For this 50 μg gapmer (SEQ ID NO: 1; 10 μg/μl) was injected under isoflurane anesthesia for 5 min. Both the left and right eye of two mice were treated (four eyes in total). Mice were sacrificed 5 days after dosing (IVT,5 dS). Control mice received the negative control gapmer (SEQ ID NO: 2), which was used in the same way. Mice were sacrificed using CO₂ asphyxiation. The corneas and retina were isolated and snap-frozen in liquid nitrogen and stored at −80° C. prior to RNA isolation that was performed as described above. MALAT1 transcripts were detected in a technical duplicate using the TaqMan assay in a one-step RT-ddPCR assay as described above.

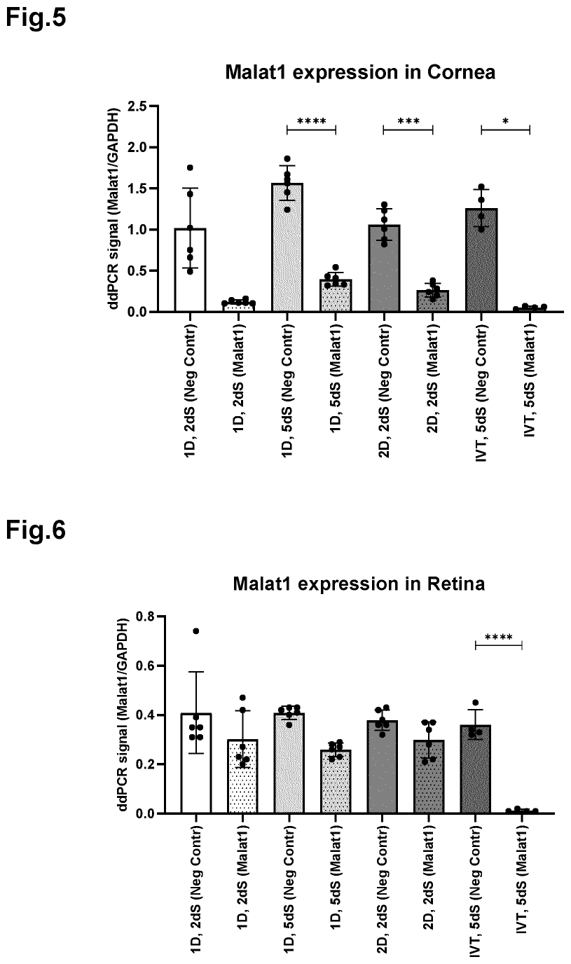

Brown-Forsythe and Welsh ANOVA tests were performed on the relative MALAT1 ddPCR signals obtained from cornea samples because the standard deviations of the different treatment groups were not equal. FIG. 5 shows the results from the cornea and indicates that a significant reduction in MALAT1 expression levels was observed in the group that received the MALAT1 gapmer compared to the group that received the negative control gapmer: 1 D,5 dS (p=<0.0001), 2 D,2 dS (p=0.0006) and IVT,5 dS (p=0.0150). Compared to the negative control the residual MALAT1 expression level in the MALAT1 gapmer was 25% in the 1 D,5 dS and 2 D,2 dS treated groups and 4% in the IVT,5dS treated group. Knockdown efficiencies were very high: 75% in the 1 D,5 dS and 2 D,5 dS treated groups and 96% in the IVT,5 dS treated group. This shows that it is possible to obtain a significant functional effect of downregulating transcript expression in the corneal layers, after applying (topically, on the outer surface of the eye) a composition comprising a nucleic acid molecule (here a gapmer) and a penetrating enhancer (here hypromellose) as well as after injecting a nucleic acid molecule (here a gapmer) by IVT administration.

An ordinary one-way ANOVA test (F=10.57) was performed on the relative MALAT1 ddPCR signals obtained from retina samples. FIG. 6 shows that only show a significant reduction in MALAT1 expression was observed in the mice that were treated intravitreally (IVT,5 dS) (p=<0.0001). Compared to the negative control the residual MALAT1 expression level in the MALAT1 gapmer was 3% in this group. Clearly, IVT is a very effective way to reach the retina with a nucleic acid molecule. But importantly, it also shows that no significant downregulation could be detected in the retina after topical administration of the nucleic acid molecule, whereas significant effects could be observed in the cornea (see above). This indicates that potential side-effects in the retina are unlikely to occur after topical administration, which adds to the safety aspect of medicaments that are targeting the corneal layers and should not reach the (healthy) retina.

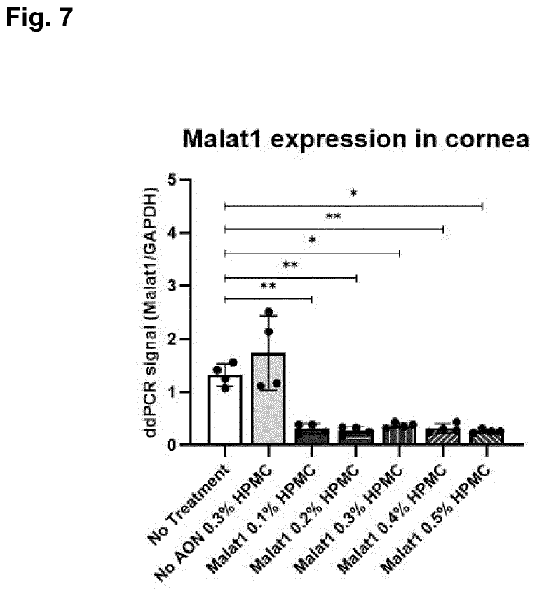

In a similar experiment as described above, a variety of HPMC concentrations was used in combination with the MALAT1-specific gapmer and non-related control gapmer to determine whether an optimal concentration would be applicable for delivery of the nucleic acid molecule to the corneal endothelium. FIG. 7 shows that all concentrations that were tested (0.1%, 0.2%, 0.3%, 0.4% and 0.5%) gave a significant downregulation of MALAT1 expression, indicating that the HPMC can be used in a range of concentrations in the compositions of the present invention.

1. An ophthalmic composition for the treatment and/or prevention of a disorder of the cornea, said composition comprising: i) a nucleic acid molecule, ii) a viscosifying polymer, and iii) a solvent, wherein the nucleic acid molecule is at least partially complementary to-, and capable of binding a target (pre-) mRNA molecule, and wherein the viscosifying polymer enables the nucleic acid molecule to penetrate through the layers within the cornea after topical administration of the composition.

2. An ophthalmic composition according to claim 1, wherein the viscosifying polymer is selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine.

3. An ophthalmic composition according to claim 1 or 2, wherein the disorder is a genetic disorder, such as a hereditary corneal dystrophy, and wherein the target (pre-) mRNA molecule is the cause of the genetic disorder.

4. An ophthalmic composition according to any one of claims 1 to 3, wherein the nucleic acid molecule comprises 10 to 40 contiguous nucleotides, preferably 10, 11, 12, 13, 14, 15, 16, 17, 18, 19, 20, 21, 22, 23, 24, 25, 26, 27, 28, 29, or 30 contiguous nucleotides.

5. An ophthalmic composition according to any one of claims 1 to 4, wherein the nucleic acid molecule is a single-stranded antisense oligonucleotide (AON) that modulates splicing of the target pre-mRNA; or that prevents or reduces RNA toxicity.

6. An ophthalmic composition of any one of claims 1 to 4, wherein the nucleic acid molecule is a gapmer that downregulates expression of the target (pre-) mRNA.

7. An ophthalmic composition according to any one of claims 1 to 6, wherein the nucleic acid molecule is non-naturally chemically modified to render it more stable towards nuclease breakdown and/or to increase its affinity towards the target (pre-) mRNA molecule.

8. An ophthalmic composition according to any one of claims 1 to 7, wherein the disorder is a dystrophy affecting the corneal epithelium, the Bowman's layer, the corneal stroma, the Descemet's membrane and/or the corneal endothelium.

9. An ophthalmic composition according to claim 8, wherein the disorder is a posterior corneal dystrophy, preferably Fuchs' Endothelial Corneal Dystrophy (FECD).

10. An ophthalmic composition according to any one of claims 1 to 6, for use in the treatment and/or prevention of a dystrophy affecting the corneal epithelium, the Bowman's layer, the corneal stroma, the Descemet's membrane and/or the corneal endothelium, preferably a posterior corneal dystrophy, more preferably FECD.

11. Use of a nucleic acid molecule and a viscosifying polymer in the manufacture of a medicament for the prevention or treatment of a dystrophy affecting the corneal epithelium, the Bowman's layer, the corneal stroma, the Descemet's membrane and/or the corneal endothelium, preferably a posterior corneal dystrophy, more preferably FECD.

12. Use according to claim 11, wherein the viscosifying polymer is selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine.

13. A method for the treatment of a disorder of the cornea, preferably a genetic disorder, said method comprising the steps of topically administering an ophthalmic composition according to any one of claims 1 to 9, allowing the entry of the corneal epithelium, the Bowman's layer, the corneal stroma, the Descemet's membrane and/or the corneal endothelium by the nucleic acid molecule, optionally allowing the entry of the cells within the corneal endothelium, and optionally allowing the passage of the nuclear membrane within the endothelium cells by the nucleic acid molecule.

14. A method of treating a disorder of the cornea, preferably a hereditary corneal dystrophy, in a mammalian subject in need thereof, comprising the steps of:
providing an ophthalmic composition comprising: i) a nucleic acid molecule, ii) a viscosifying polymer, and iii) a solvent, wherein the nucleic acid molecule is at least partially complementary to a target (pre-) mRNA molecule causing the disorder;
administering the ophthalmic composition topically to the anterior side of one or both corneas of the subject;
allowing the entry of the nucleic acid molecule to a diseased cell within the corneal epithelium, the Bowman's layer, the corneal stroma, the Descemet's membrane and/or the corneal endothelium; and
allowing the nucleic acid molecule to hybridize to a complementary sequence of a (pre-) mRNA molecule within the diseased cell.

15. A method according to claim 14, wherein the nucleic acid molecule modulates the splicing of the (pre-) mRNA, prevents or diminishes the formation of RNA foci, or wherein the nucleic acid molecule causes a nuclease-dependent breakdown of the (pre-) mRNA.

16. A method according to claim 13 or 14, wherein the viscosifying polymer is selected from the group consisting of: hydroxypropyl methylcellulose (HPMC), hydroxypropyl cellulose, methylcellulose, carbomer, hyaluronan, chitosan, N-trimethyl chitosan, N-carboxymethyl chitosan, Na carboxymethylcellulose, polygalacturonic acid, Na alginate, xanthan gum, xyloglucan gum, scleroglucan, polyvinyl alcohol, and polyvinyl pyrrolidine.

17. Use of an ophthalmic composition for topical administration to the eye of a mammalian subject suffering from a disorder of the cornea, preferably a hereditary corneal dystrophy, wherein the composition comprises: i) a nucleic acid molecule, ii) a viscosifying polymer, and iii) a solvent, wherein the nucleic acid molecule is at least partially complementary to a target (pre-) mRNA molecule causing the disorder, and wherein the viscosifying polymer enables the nucleic acid molecule to penetrate through the different layers of the cornea.


SEQUENCE LISTING (2 sequences)

SEQ ID NO: 1 - DNA, 16 bp, Artificial sequence
  Mouse Malat1 gapmer
gtcacaatgcattcta

SEQ ID NO: 2 - DNA, 16 bp, Artificial sequence
  Control gapmer
gctcccttcaatccaa